(19) United States
(12) Patent Application Publication
(10) Pub. No.: US 2017/0151157 A1
(43) Pub. Date: Jun. 1, 2017

(54) CLEAR VOLUMIZING SHAMPOO CONTAINING STYLING AND CONDITIONING POLYMERS AND FILOXANE (APTES-AMINOPROPYL TRIETHOXYSILANE)

(71) Applicant: L'OREAL, Paris (FR)

(72) Inventors: Halil GEVGILILI, Weehawken, NJ (US); Eva Eliza ZION, Bridgewater, NJ (US); Adolfo R. PERTUZ, West New York, NJ (US); Jun LIANG, Brooklyn, NY (US)

(21) Appl. No.: 14/953,145

(22) Filed: Nov. 27, 2015

(51) Int. Cl. A61K 8/81; A61Q 5/12; A61Q 5/06; A61Q 5/02; A61K 8/58

(52) U.S. Cl. CPC A61K 8/8147; A61Q 5/02; A61K 8/585; A61Q 5/06; A61Q 5/12; A61K 2800/262

(57) ABSTRACT

Disclosed are compositions for washing, cleansing, and/or rinsing keratin substrates, comprising: (a) an ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type; (b) a copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole; (c) a silane; (d) a surfactant mixture; and (e) water. Also disclosed are processes for washing, cleansing, and/or rinsing keratin substrates using the composition, and processes for imparting instant and long lasting volume as well as conditioning to the keratin substrates.

### FIELD OF THE INVENTION

The present application relates to cosmetic compositions for use on keratinous substrates, such as keratin fibers. In particular, it relates to compositions and methods for washing and/or cleansing hair.

### BACKGROUND

Certain hair types are naturally thin and/or fine. In addition, any type of hair can diminish in quality and/or quantity over time by age and/or due to factors such as natural greasiness, sweat, shredded skin cells from the scalp, pollution, and dirt. These factors can result in thinning hair and/or harm the visual appearance and the feel of the hair, and lead to lank body and decreased volume. The magnitude of the consequences of these factors, which are almost all inevitable, is variable, depending on, for example, the quality of the hair, length, style, and environmental factors.

Shampoos are used to combat these drawbacks. Conventional cleansing compositions such as shampoos, for example, contain standard surfactants such as anionic, nonionic and/or amphoteric type surfactants. These compositions can be applied onto a wet keratinous substrate and the lather they generate make it possible, after rinsing with water, to remove the diverse types of soils typically present on the substrate such as hair and/or skin.

These cleansing compositions, while providing good cleansing power, may yield poor intrinsic cosmetic properties due to the fact that the nature of such a cleansing treatment may result in a less conditioned or rough feel to the hair of the hair due to, for example, the gradual removal of the natural or applied fats, lipids, or proteins contained in or at the surface of the hair.

Shampoos and cleansing compositions sometimes include polymers as thickeners, for example cellulose or cellulose derivatives, different type gums or acrylate-based cross-linked polymers such as carbomers and carbopols. In addition, shampoos and cleansing compositions sometimes include cationic polymers such polyquaternium polymers and silicones to provide or enhance cosmeticity effects. However, the use of polymers with varying ionicities can result in unstable compositions.

Styling products, for example gels and mousses, are leave-in compositions that may provide volume and body while in the hair. Some leave-in styling products use polymers, for example film-forming polymers, to provide volumizing properties. However, some polymers can be easily removed from the hair, for example by rinsing or washing. Thus, any cosmetic benefits to the hair from such products are generally diminished or removed once the hair is rinsed or washed.

Thus the present disclosure relates to personal cleansing and/or washing compositions. More particularly, the embodiments of the disclosure relate to rinse-off compositions that provide volumizing properties on keratinous substrates, such as keratin fibers, in particular hair, wherein the volumizing effects on hair can be long-lasting, that is, the effect remains even after repeated washings or cleansing using the compositions of the present disclosure. The disclosure also relates to a hair cleansing and/or washing/rinsing process using this composition.

It is also an object of embodiments of the disclosure to provide a stable rinse-off cleansing and/or washing composition that cleans a keratinous substrate while imparting increased mass, body or volume and maintaining good deposition and film formation capability on hair fibers to provide excellent volumizing and cosmetic properties, for example long-lasting volume, conditioning, softness and detangling.

Another object of the embodiments of the disclosure is to provide rinse-off cleansing and/or washing compositions that are clear in appearance, an attribute that is desired by consumers.

According to embodiments of the disclosure, a rinse-off cleansing and/or washing composition comprising a) an ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type; b) a copolymer comprising a monomer (i) selected from acrylic acid amide, methacrylic acid amide, N-alkylacrylic acid amide and N-alkyl-methacrylic acid amide, a monomer (ii) selected from N-vinyllactams, a monomer (iii) selected from quaternized N-vinylimidazoles and the monomer N-vinylimidazole; c) a silane; d) a surfactant mixture; and e) water. It has now been surprisingly and unexpectedly discovered that such a composition is stable, it is clear, and delivers instant as well as long lasting mass, body, and/or volume to the treated hair. Hair cleansed and/or washed with the rinse-off compositions according to embodiments of the disclosure has improved mass, body, and volume.

Also disclosed is a process for washing, cleansing, and/or rinsing keratinous substrates such as the hair and/or the scalp using the composition according to embodiments of the disclosure, the process including applying to the keratinous substrates, a composition as defined above, rinsing off the composition, and optionally drying said keratinous substrates.

Other subjects, characteristics, aspects and advantages of embodiments of the disclosure will emerge even more clearly on reading the description and the various examples that follow.

### BRIEF SUMMARY

Embodiments of the disclosure relate to a rinse-off composition for cleansing keratinous substrates, the composition comprising:

(a) at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;

(b) at least one copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole;

(c) at least one silane corresponding to formula (I):

R₁Si(OR₂)_{z}(R₃)ₓ(OH)_{y} (I)

in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3; and

(d) a surfactant mixture comprising:
i. at least one amphoteric surfactant;
ii. at least one nonionic surfactant; and
iii. at least one anionic surfactant;
   wherein the total amount of amphoteric surfactants is about 20% or more by weight, based on the total weight of the surfactant mixture; and

(e) water.

Embodiments of the disclosure also relate to a process for washing, cleansing, and/or conditioning a keratin substrate, involving applying the above-described composition onto the keratin substrate, and to methods of increasing the volume of the hair by washing, cleansing, and/or conditioning with the above-described composition.

The rinse-off compositions of embodiments of the disclosure may be stable over time and not undergo phase separation.

In certain embodiments of the disclosure, the rinse-off compositions are clear.

It is to be understood that both the foregoing general description and the following detailed description are exemplary and explanatory only, and are not restrictive of the disclosure.

### DETAILED DESCRIPTION

As used herein, the expression “at least one” means one or more and thus includes individual components as well as mixtures/combinations.

In the present patent application, a species is termed as being “anionic” when it bears at least one permanent negative charge or when it can be ionized as a negatively charged species, under the conditions of use of the compositions of embodiments of the disclosure (for example the medium or the pH) and not comprising any cationic filler.

In the present patent application, a species is termed as being “cationic” when it bears at least one permanent positive charge or when it can be ionized as a positively charged species, under the conditions of use of the compositions of embodiments of the disclosure (for example the medium or the pH) and not comprising any anionic filler.

A species is termed as being “nonionic” when it is neither cationic nor anionic within the meaning of the disclosure, in particular when it comprises no cationic or anionic groups within the meaning of the disclosure.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%), such as within 9%, 8%, 7%, 6%, 5%, 4%, 3%, 2%, or 1%, according to various embodiments.

The term “comprising” (and its grammatical variations) as used herein is used in the inclusive sense of “having” or “including” and not in the exclusive sense of “consisting only of”.

“Keratinous substrates” as used herein, includes, but is not limited to keratin fibers such as hair on the human head and hair comprising eyelashes. “Keratinous substrates” as used herein, may also refer to the skin such as lips, finger nails or toe nails, and the scalp.

As used herein, the terms “applying a composition onto “keratinous substrates” as used herein, includes, and “applying a composition onto “keratinous substrates” or “keratin fibers” such as hair on a human head with at least one of the compositions of the disclosure, in any manner.

As used herein, “formed from,” means obtained from chemical reaction of, wherein “chemical reaction,” includes spontaneous chemical reactions and induced chemical reactions. As used herein, the phrase “formed from,” is open ended and does not limit the components of the composition to those listed.

The term “stable” as used herein means that the composition does not exhibit phase separation and/or crystallization.

The term “treat” (and its grammatical variations) as used herein refers to the application of the compositions of the present disclosure onto keratin fibers such as hair.

“Volatile”, as used herein, means having a flash point of less than about 100° C.

“Non-volatile”, as used herein, means having a flash point of greater than about 100° C.

“Substituted” as used herein, means comprising at least one substituent. Non-limiting examples of substituents include atoms, such as oxygen atoms and nitrogen atoms, as well as functional groups, such as acyloxyalky groups, carboxylic acid groups, amine or amino groups, acylamino groups, amide groups, halogen containing groups, ester groups, thiol groups, sulphonate groups, thiosulphate groups, siloxane groups, and polysiloxane groups. The substituent(s) may be further substituted.

The terms “organic compound” and “having an organic structure” mean compounds containing carbon atoms and hydrogen atoms and optionally heteroatoms such as S, O, N or P, alone or in combination.

The term “polymer” is understood to mean, within the meaning of the disclosure, a compound characterized by the multiple repetition of one or more species of atoms or groups of atoms, known as monomers, linked to each other in amounts sufficient to provide a set of properties that do not vary markedly with the addition or removal of one or a few of the monomers.

The term “film-forming polymer” is understood to mean a polymer which is capable of forming, by itself alone or in the presence of an additional film-forming agent, a macroscopically continuous or semi-continuous film on a support, in particular on keratinous substances, such as a cohesive film.

The term “rinse-off” is used herein to mean that keratinous substrate such as hair is rinsed and/or washed either after or during the application of the composition, and before drying and/or styling said keratinous substrate. At least a portion of the composition is removed from the keratinous substrate during the rinsing and/or washing. A “leave-on” product refers to a cosmetic composition such as a hair care composition that is applied to a keratinous substrate such as hair and not further subjected to a rinsing and/or washing step before drying and/or styling the substrate.

The compositions and methods of the present disclosure can comprise, consist of, or consist essentially of the essential elements and limitations of the disclosure described herein, as well as any additional or optional ingredients, components, or limitations described herein or otherwise useful.

In an embodiment of the present disclosure, the rinse-off composition for cleansing and/or washing keratinous substrates comprises:

(a) at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;

(b) at least one copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole;

(c) at least one silane corresponding to formula (I):

R₁Si(OR₂)_{z}(R₃)ₓ(OH)_{y} (I)

in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, preferably from 1 to 3, and even more preferably, z=3; and
x denotes an integer ranging from 0 to 2, with z+x+y=3; and

(d) a surfactant mixture comprising:
i. at least one amphoteric surfactant;
ii. at least one nonionic surfactant; and
iii. at least one anionic surfactant;
   wherein the total amount of amphoteric surfactants is about 20% or more by weight, based on the total weight of the surfactant mixture; and

(e) water.

In another embodiment of the present disclosure, the rinse-off composition for cleansing and/or washing keratinous substrates comprises:
(a) from about 0.1% to about 0.2% by weight, based on the total weight of the composition, of at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
(b) from about 0.05% to about 0.08% by weight, based on the total weight of the composition, of at least one copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole;
(C) from about 0.5% to about 0.75% by weight, based on the total weight of the composition, of at least one silane corresponding to formula (I):

R₁Si(OR₂)_{z}(R₃)ₓ(OH)_{y} (I)

in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, preferably from 1 to 3, and even more preferably, z=3; and
x denotes an integer ranging from 0 to 2, with z+x+y=3; and
(d) a surfactant mixture comprising:
   i. at least one amphoteric surfactant;
   ii. at least one nonionic surfactant; and
   iii. at least one anionic surfactant;
      wherein the total amount of amphoteric surfactants is about 20% or more by weight, based on the total weight of the surfactant mixture; and
(e) water.

In other embodiments, the anionic surfactant is chosen from sodium laureth sulfate, sodium lauryl sulfate, sodium lauroyl methyl isethionate, and mixtures thereof.

In some other embodiments, the amphoteric surfactant is chosen from (C8-C20)alkylbetaines, (C8-C20)alkylamido(C1-C6)alkylbetaines, (C8-C20)alkylamphoacetates and (C8-C20)alkylamphodiacetates, and mixtures thereof, and preferably from coco-betaine, cocamidopropyl betaine, cocoamphoacetate, cocoamphodiacetate, and their salts, and mixtures thereof.

In yet other embodiments, the nonionic surfactant is chosen from alkyl polyglucosides, fatty acid alkanolamides, alkoxylated alcohols, and mixtures thereof, and preferably from coco-glucoside, cocamide MIPA, PEG-40 hydrogenated castor oil, and mixtures thereof.

In certain embodiments, the total amount of nonionic surfactants is about 8% or more by weight, based on the total weight of the surfactant mixture in the composition of the present disclosure.

In yet other embodiments, the ampholytic polymer (a) is polyquaternium-53 and the copolymer (b) is polyquaternium-68.

In other embodiments, the silane is an alkoxysilane, preferably chosen from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane or their oligomers thereof, and mixtures thereof.

In certain embodiments, the rinse-off/cleansing composition of the present disclosure comprises an ampholytic polymer (a) chosen from polyquaternium-53, a copolymer (b) chosen from polyquaternium-68, a silane (c) of formula (1) chosen from an alkoxysilane having one silicon atom and wherein R₁ is an aminoalkyl group, R₂ is a C1 to C6 alkyl group, and z=3 and x=y=0, a surfactant mixture comprising anionic, amphoteric, and nonionic surfactants, and water, wherein the composition is clear and the composition is a shampoo. Preferably, the silane is 3-aminopropyltriethoxysilane (APTES).

In other embodiments, the total amount of nonionic surfactants is about 8% or more by weight, based on the total weight of the surfactant mixture in the composition of the present disclosure.

The compositions according to various embodiments of the disclosure have a homogenous texture, i.e., not lumpy, and are easy to apply and spread on the hair.

It has been surprisingly and unexpectedly discovered that the compositions according to the disclosure are stable over time, exhibit no visible phase separation, appear clear in appearance, and allow retention of the cosmetic effects of the at least one copolymer (b) and the at least ampholytic polymer (a), such that hair is effectively or satisfactorily volumized and easy to detangle and comb through after treatment with the composition. It is possible that the volumizing effect imparted to the hair remains even after several washings/cleansings of the hair.

### Ampholytic Polymer (a)

Embodiments of the disclosure employ at least one ampholytic polymer chosen from those that contain units comprising primary, secondary, tertiary and/or quaternary amine groups that may either form part of the main polymer chain or may be borne by a side substituent directly connected thereto.

In certain embodiments, the ampholytic polymer can be pH adjusted to become cationic polymers.

Thus, the composition according to the present invention contains one or more amphoteric polymers comprising a repetition of:

(i) one or more units obtained from a monomer of (meth)acrylamide type,
(ii) one or more units obtained from a monomer of (meth)acrylamidoalkyltrialkylammonium type, and
(iii) one or more units obtained from an acidic monomer of (meth)acrylic acid type.

Preferably, the units derived from a monomer of (meth)acrylamide type (i) of the amphoteric polymer are units of structure (I) below:

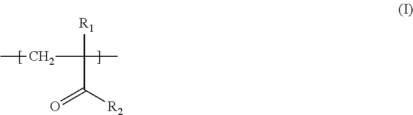

in which:

R1 denotes H or CH3,

R2 is chosen from an amino, dimethylamino, tert-butylamino or dodecylamino radical, or —NH—CH2OH.

Preferably, the amphoteric polymer of the invention comprises a repetition of only one unit of formula (I).

The unit derived from a monomer of (meth)acrylamide type of formula (I) in which R1 denotes H and R2 is an amino radical is particularly preferred. It corresponds to the acrylamide monomer per se.

Preferably also, the units derived from a monomer of (meth)acrylamidoalkyltrialkylammonium type (ii) of the amphoteric polymer are units of structure (II) below:

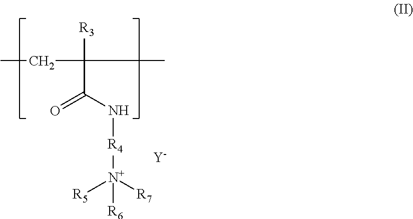

in which:

R3 denotes H or CH3,

R4 denotes a group (CH2)k with k being an integer ranging from 1 to 6 and preferably from 2 to 4;

R5 and R6, and R7, which may be identical or different, each denote an alkyl group containing from 1 to 4 carbon atoms;

Y— is an anion such as bromide, chloride, acetate, borate, citrate, tartrate, bisulfate, bisulfite, sulfate or phosphate.

Among these units derived from a monomer of (meth)acrylamidoalkyltrialkylammonium the ones that are preferred are those derived from the methacrylamidopropyltrimethylammonium chloride monomer, which R3 denotes a methyl radical, k is equal to 3, R5, R6 and R7 denote a methyl radical, and Y— denotes a chloride anion.

Preferably, the amphoteric polymer of the invention comprises a repetition of only one unit of formula (II).

Finally, the units derived from an acidic monomer of (meth)acrylic acid type (iii) of the amphoteric polymer are preferentially chosen from the units of formula (III):

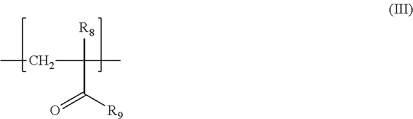

in which:

R8 denotes H or CH3,

R9 denotes a hydroxyl radical or a radical —NH—C(CH3)2-CH2-SO3H. The preferred units of formula (III) correspond to the acrylic acid, methacrylic acid and 2-acrylamino-2-methylpropanesulfonic acid monomers.

Preferably, the unit derived from an acidic monomer of (meth)acrylic acid type is that derived from acrylic acid, for which R8 denotes a hydrogen atom and R9 denotes a hydroxyl radical.

The acidic monomer(s) of (meth)acrylic acid type may be non-neutralized or partially or totally neutralized with an organic or mineral base.

Preferably, the amphoteric polymer of the invention comprises a repetition of only one unit of formula (III).

According to one preferred embodiment of the invention, the amphoteric polymer(s) comprise at least 30 mol percent of units derived from a monomer of (meth)acrylamide type. Preferably, they comprise from 30 mol percent to 70 mol percent and more preferably from 40 mol percent to 60 mol percent of units derived from a monomer of (meth)acrylamide type

The contents of units derived from a monomer of (meth)acrylamidoalkyltrialkylammonium type may advantageously be the following: from 10 mol percent to 60 mol percent and preferentially from 20 mol percent to 55 mol percent.

The contents of units derived from an acidic monomer of (meth)acrylic acid type may advantageously be the following: from 1 mol percent to 20 mol percent and preferentially from 5 mol percent to 15 mol percent.

According to one preferred embodiment of the invention, the amphoteric polymer comprises:

from 30 mol percent to 70 mol percent and more preferably from 40 mol percent to 60 mol percent of units derived from a monomer of (meth)acrylamide type,

from 10 mol percent to 60 mol percent and preferentially from 20 mol percent to 55 mol percent of units derived from a monomer of (meth)acrylamidoalkyltrialkylammonium type;

from 1 mol percent to 20 mol percent and preferentially from 5 mol percent to 15 mol percent of units derived from an acidic monomer of (meth)acrylic acid type.

The amphoteric polymer(s) according to the present invention may also comprise additional units, other than the units derived from a monomer of (meth)acrylamide type, of (meth)acrylamidoalkyltrialkylammonium type and of (meth)acrylic acid type.

According to one preferred embodiment of the invention, the amphoteric polymer(s) consist solely of units derived from monomers (i) of (meth)acrylamide type, (ii) of (meth)acrylamidoalkyltrialkylammonium type and (iii) of (meth)acrylic acid type.

As examples of amphoteric polymers that are particularly preferred, mention may be made of acrylamide/methylacrylamidopropyltrimethylammonium chloride/acrylic acid terpolymers. Such polymers are listed in the CTFA dictionary. International Cosmetic Ingredient Dictionary, 10th edition 2004, under the name Polyquaternium 53. Corresponding products are especially sold under the names Merquat 2003 or Merquat 2003PR by the company (LUBRIZOL).

The amphoteric polymer according to the invention may conventionally be prepared by polymerization starting with its various monomers, according to techniques known to those skilled in the art and especially by radical polymerization.

In certain embodiments, the at least one ampholytic polymer is an ampholytic terpolymer consisting of acrylic acid, methacrylamidopropyl trimethyl ammonium chloride. According to some embodiments, the ampholytic polymer is an ampholytic terpolymer known by the INCI name of polyquaternium-53 (commercially available from Nalco (Lubrizol), under the tradename, Merquat™ 2003 PR Polymer, 19.5% to 22.5% by weight active).

In some embodiments, the ampholytic polymer has a monomer distribution such that the overall charge of the ampholytic polymer is moderately to highly cationic. For example, the ampholytic polymer may comprise about 0-20% anionic monomer, about 25-80% cationic monomer, and about 0-70% nonionic monomer. In certain embodiments, the ampholytic polymer comprises about 5-15% anionic monomer, about 30-60% cationic monomer, and about 40-60% nonionic monomer.

According to various embodiments, the ampholytic polymer that may be chosen has a cationic charge density optimized to provide desired properties to the composition. Thus, in certain embodiments, the ampholytic polymer has a moderate charge density. Without wishing to be bound by theory, it is believed that in some embodiments, an ampholytic polymer having a moderate charge density contributes to achieving clarity and stability of the composition of the invention.

In some embodiments, the cationic charge density of the ampholytic polymer ranges from greater than about 1.0 meq/g to about 3.5 meq/g, such as about 1.8 meq/g to about 3.1 meq/g, about 2.0 meq/g to about 2.8 meq/g, about 2.0 meq/g to about 3.1 meq/g, about 2.4 meq/g to about 3.1 meq/g, or about 2.0 meq/g to about 2.5 meq/g. This charge density may be determined either by calculation from the structure of the polymer or experimentally via the Kjeldahl method. In some embodiments, the charge density of the ampholytic polymer may vary, depending on the pH, which is between about 5 and about 7 in certain embodiments. In some other embodiments, the charge density of the ampholytic polymer is between about 5 and about 6. In one embodiment, the ampholytic polymer has a cationic charge density of about 2.4 meq/g to about 3.1 meq/g. One such ampholytic polymer having a charge density of about 2.4 meq/g to about 3.1 meq/g is polyquaternium-53.

In some embodiments, the molecular weight of the ampholytic polymer affects the volumizing and/or conditioning properties of the composition. In certain embodiments, the volumizing properties of the composition are at least in part determined by film formation, which is in turn at least in part influenced by the molecular weight of the cationic polymer.

According to various embodiments, the ampholytic polymer that may be chosen may have a weight average molecular weight ranging from about 500,000 to about 3,000,000, such as about 1,000,000 to about 2,000,000, or about 1,200,000 to about 1,500,000, including all ranges and subranges therebetween. In one embodiment, the ampholytic polymer has a molecular weight of about 1,200,000 to about 1,500,000.

The total amount of the ampholytic polymer may range from about 0.1% to about 5% by weight, based on the total weight of the composition, including all ranges and subranges therebetween.

In certain embodiments, the total amount of the ampholytic polymer ranges from about 0.05% to about 0.4% by weight, such as from about 0.07% to about 0.35% by weight, from about 0.08% to about 0.3% by weight, from about 0.09% to about 0.25% by weight, or from about 0.1% to about 0.2%, by weight of active material, based on the total weight of the composition, including all ranges and subranges therebetween.

In various embodiments, the total amount of the ampholytic polymer is about 0.05%, 0.06%, 0.07%, 0.075%, 0.08%, 0.09%, 0.1%, 0.11%, 0.12%, 0.13%, 0.14%, 0.15%, 0.16, 0.175%, 0.18%, 0.19%, 0.2%, 0.225%, 0.25%, 0.275%, 0.3%, 0.325%, 0.35%, 0.375%, and 0.4%, by weight of active material, based on the total weight of the composition.

### Copolymer (b)

The copolymer (b) of the present disclosure comprises:
at least one monomer (i) selected from acrylic acid amide, methacrylic acid amide, N—C1-C10-alkylacrylic acid amide and N—C1-C10-alkylmethacrylic acid amide,
at least one monomer (ii) selected from N-vinyllactams and N-vinylpyrrolidone,
at least one monomer (iii) selected from quaternized N-vinylimidazoles, and
a monomer selected from N-vinylimidazole.

Copolymers of the disclosure which are made up from the aforementioned monomers are understood in the sense of the present invention to be only those copolymers which contain, in addition to polymer units resulting from the incorporation of the aforementioned monomers (i), (ii), and (iii), and N-vinylimidazole into the copolymer, maximum 5 wt percent, preferably maximum 1 wt percent polymer units attributed to the incorporation of other monomers. The copolymers are preferably synthesized exclusively from polymer units which result from the incorporation of the aforementioned monomers (i), (ii), and (iii), and N-vinylimidazole into the copolymer.

The copolymer (b) of the present invention is a cationic polymer.

Preferred monomers (i) are acrylic acid amide and methacrylic acid amide, with methacrylic acid amide being especially preferred.

Preferred monomers (ii) are N-vinylcaprolactam and N-vinylpyrrolidone, with N-vinylpyrrolidone being especially preferred.

Preferred monomers (iii) are salts of 3-alkyl-1-vinylimidazolium, in particular 3-(C1-C10-alkyl)-1-vinylimidazolium with physiologically acceptable anions. Suitable physiologically acceptable anions include in particular halides such as chloride, bromide and iodide, bicarbonate, bisulfate, monoalkyl sulfate, in particular monomethyl sulfate and dihydrogen phosphate. Preferred physiologically tolerable anions include chloride and monomethyl sulfate.

Monomer (iii) is particularly preferably 3-methyl-1-vinylimidazolium methyl sulfate.

An especially preferred copolymer of the disclosure is made up from:
at least one monomer (i) selected from acrylic acid amide and methacrylic acid amide,
at least one monomer (ii) selected from N-vinylcaprolactam and N-vinyl pyrrolidone,
at least one monomer (iii) selected from 3-methyl-1-vinylimidazolium methyl sulfate and
N-vinylimidazole.

The copolymers of the disclosure can be synthesized from the aforementioned monomers by means of the known polymerization methods.

Most especially preferred copolymers of the embodiments of the disclosure are the copolymers that are known as polyquaternium-68 according to INCI nomenclature and are synthesized from vinylpyrrolidone, methacrylic acid amide, vinylimidazole and 3-methyl-1-vinylimidazolium methyl sulfate. These are commercially available under the brand name Luviquat(R) Supreme AT 1 from the supplier BASF.

The compositions of the disclosure preferably contain the copolymer (b) in an amount of from about 0.005% to about 0.2% by weight, such as from about 0.01% to about 0.15% by weight, from about 0.02% to about 0.12% by weight, from about 0.04% to about 0.1% by weight, or from about 0.05% to about 0.08%, by weight of active material, based on the total weight of the composition, including all ranges and subranges therebetween.

In various embodiments, the total amount of the copolymer (b) is about 0.005%, 0.0075%, 0.01%, 0.015%, 0.02%, 0.025%, 0.03%, 0.035%, 0.04%, 0.045%, 0.05%, 0.055%, 0.06%, 0.07%, 0.08%, 0.09%, 0.1%, 0.12%, 0.14%, 0.15%, 0.16%, 0.175%, 0.18%, 0.2%, by weight of active material, based on the total weight of the composition.

### Silane

According to the invention, the composition comprises one or more silanes corresponding to formula (I) and/or their oligomers thereof.

Formula (I) is a follows:

R₁Si(OR₂)_{z}(R₃)ₓ(OH)_{y} (I)

in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3.

The term “oligomer” means the polymerization products of the compounds of formula (I) comprising from 2 to 10 silicon atoms.

Preferably, R₁ is a linear or branched, preferably linear, saturated C₁-C₂₂, in particular C₂-C₁₂, hydrocarbon-based chain, which may be substituted with an amine group NH₂ or NHR (R═C₁-C₂₀, in particular C₁-C₆, alkyl).

Preferably, R₂ represents an alkyl group comprising from 1 to 4 carbon atoms, better still a linear alkyl group comprising from 1 to 4 carbon atoms, and preferably the ethyl group.

Preferably, z ranges from 1 to 3.

Preferably, y=0.

Preferentially, z=3, and therefore x=y=0.

In one embodiment of the invention, R₁ represents a linear alkyl group comprising from 7 to 18 carbon atoms and more particularly from 7 to 12 carbon atoms, or a C₁-C₆, preferably C₂-C₄, aminoalkyl group. More particularly, R₁ represents an octyl group.

In one embodiment of the invention, R₁ is a linear or branched, saturated or unsaturated C₁-C₂₂ hydrocarbon-based chain, substituted with an amine group NH₂ or NHR (R═C₁-C₂₀, in particular C₁-C₆, alkyl, C3-C₄₀ cycloalkyl or C₆-C₃₀ aromatic). In this variant, R₁ preferably represents a C₁-C₆, preferably C₂-C₄, aminoalkyl group.

Preferably, the at least one silane of the present invention is an alkoxysilane.

Preferably, the composition comprises at least one compound of formula (I) chosen from alkoxysilanes such as 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane, N-(2-aminoethylaminomethyl)phenethyltrimethoxysilane, and mixtures thereof.

In certain embodiments, the siliane of the disclosure is an alkoxysilane selected from 3-aminopropyltriethoxysilane (APTES), 3-aminoethyltriethoxysilane (AETES), 3-aminopropylmethyldiethoxysilane and N-(2-aminoethyl)-3-aminopropyltriethoxysilane or oligomers thereof, and mixtures thereof.

In preferred embodiments, the silane is chosen from 3-aminopropyltriethoxysilane (APTES) or oligomers thereof, or mixtures thereof.

The silanes used in the composition of the invention, in particular those comprising a basic function, may be partially or totally neutralised in order to improve the water-solubility thereof. In particular, the neutralising agent may be chosen from organic or inorganic acids, such as citric acid, tartaric acid, lactic acid or hydrochloric acid.

Preferably, the optionally neutralised silanes according to the invention are water-soluble and in particular soluble at a concentration of 2%, better still at a concentration of 5% and even better still at a concentration of 10% by weight in water at a temperature of 25° C. and at atmospheric pressure (1 atm). The term “soluble” is intended to mean the formation of a single macroscopic phase.

The silane(s) of formula (I) and/or oligomers thereof may be present in the composition according to the invention in an amount of about 0.05% to less than 1% by weight, such as from about 0.1% to about 0.95% by weight, from about 0.5% to about 0.85% by weight, from about 0.55% to about 0.8% by weight, or from about 0.5% to about 0.75%, by weight of active material, based on the total weight of the composition, including all ranges and subranges therebetween.

In various embodiments, the total amount of the silane is about 0.05%, 0.075%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, and 0.95% by weight of active material, based on the total weight of the composition.

### Surfactant Mixture

The composition of the disclosure comprises a surfactant mixture comprising amphoteric (zwitterionic) surfactants and nonionic surfactants.

### Amphoteric or Zwitterionic Surfacant

The composition according to the invention comprises one or more amphoteric surfactants.

The amphoteric surfactants that may be used in the invention may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C8-C20)alkylbetaines, sulfobetaines, (C8-C20)alkylsulfobetaines, (C8-C20)alkylamido(C1-C6)alkylbetaines, such as cocamidopropylbetaine, and (C8-C20)alkylamido(C1-C6)alkylsulfobetaines, and mixtures thereof.

Among the optionally quaternized secondary or tertiary aliphatic amine derivatives that may be used, mention may also be made of the products of respective structures (A1) and (A2) below:

Rₐ—CON(Z)CH₂—(CH₂)ₘ—N⁺(R_{b})(R_{c})(CH₂COO⁻) (A1)

wherein:

Rₐ represents a C₁₀-C₃₀ alkyl or alkenyl group derived from an acid Rₐ—COOH preferably present in hydrolysed coconut oil, a heptyl group, a nonyl group or an undecyl group,

R_{b} represents a β-hydroxyethyl group,

R_{c} represents a carboxymethyl group;

m is equal to 0, 1 or 2,

Z represents a hydrogen atom or a hydroxyethyl or carboxymethyl group;

Rₐ—CON(Z)CH_{2—}(CH₂)ₘ—N(B)(B′) (A2)

wherein:

B represents —CH₂CH₂OX′, with X′ representing —CH₂—COOH, CH₂—COOZ′, —CH₂CH₂—COOH, —CH₂CH₂—COOZ′, or a hydrogen atom,

B′ represents —(CH₂)_{z}—Y′, with z=1 or 2, and Y′ representing —COOH, —COOZ′, —CH₂—CHOH—SO₃H or —CH₂—CHOH—SO₃Z′,

m′ is equal to 0, 1 or 2,

Z represents a hydrogen atom or a hydroxyethyl or carboxymethyl group,

Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane,

R_{a′} represents a C₁₀-C₃₀ alkyl or alkenyl group of an acid R_{a′}COOH preferably present in hydrolysed linseed oil or coconut oil, an alkyl group, in particular a C₁₇ alkyl group, and its iso form, or an unsaturated C₁₇ group.

The compounds corresponding to formula (A2) are preferred.

Among the compounds corresponding to formula (A2) in which X′ represents an hydrogen atom, mention may be made of compounds classified in the CTFA dictionary, under the names sodium cocoamphoacetate, sodium lauroamphoacetate, sodium caproamphoacetate and sodium capryloamphoacetate.

Other compounds corresponding to formula (A2) are disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

Examples that may be mentioned include the cocoamphodiacetate sold by the company Rhodia under the trade name Miranol® C2M Concentrate, the sodium cocoamphoacetate sold under the trade name Miranol Ultra C 32 and the product sold by the company Chimex under the trade name CHIMEXANE HA.

Use may also be made of the compounds of formula (A3):

R_{a′} NH—CH(Y″)—(CH₂)n-C(O)—NH—(CH₂)n′-N(R_{d})(Rₑ) (A3)

wherein:
R_{a″} represents a C₁₀-C₃₀ alkyl or alkenyl group of an acid R_{a′}—C(O)OH preferably present in hydrolysed linseed oil or coconut oil;
Y″ represents the group —C(O)OH, —C(O)OZ″, —CH₂—CH(OH)—SO₃H or the group —CH₂—CH(OH)—SO₃—Z″, with Z″ representing a cationic counterion resulting from an alkali metal or alkaline-earth metal, such as sodium, an ammonium ion or an ion resulting from an organic amine;
R_{d} and Rₑ represent, independently of each other, a C1-C4 alkyl or hydroxyalkyl radical; and
n and n′ denote, independently of each other, an integer ranging from 1 to 3.

Among the compounds corresponding to formula (A3), mention may in particular be made of the compound classified in the CTFA dictionary under the name sodium diethylaminopropyl cocoaspartamide, such as the one sold by the company Chimex under the name CHIMEXANE HB.

Preferably, the amphoteric surfactants are chosen from (C8-C20)alkylbetaines, (C8-C20)alkylamido(C1-C6)alkylbetaines, (C8-C20)alkylamphoacetates and (C8 -C20)alkylamphodiacetates, and mixtures thereof.

In preferred embodiments of the disclosure, the amphoteric surfactants are chosen from coco-betaine sold by BASF as DEHYTON AB 30

The total amount of amphoteric and/or zwitterionic surfactant present in the composition is from about 3% to 20% by weight, such as from about 3% to about 15% by weight, from about 3% to about 10% by weight, from about 3.5% to about 8% by weight, or from about 4% to about 6%, by weight of active material, based on the total weight of the composition, including all ranges and subranges therebetween.

In various embodiments, the total amount of amphoteric and/or zwitterionic surfactant is about 3%, 3.5%, 4%, 4.5%, 5%, 5.1%, 5.2%, 5.3%, 5.4%, 5.5%, 5.6%, 5.7%, 5.8%, 5.9%, 6%, 6.5%, 7%, 7.5%, 8%, 8.5%, 9%, 9.5%, 10%, 12%, 14%, 15%, 18%, and 20%, by weight of active material, based on the total weight of the composition.

### Non-Ionic Surfactant

Examples of nonionic surfactants that may be used in the composition according to embodiments of the disclosure are described, for example, in the Handbook of Surfactants by M. R. Porter, published by Blackie & Son (Glasgow and London), 1991, pp. 116-178. They are especially chosen from alcohols, α-diols and (C₁-C₂₀)alkylphenols, these compounds being polyethoxylated, polypropoxylated and/or polyglycerolated, the number of ethylene oxide and/or propylene oxide groups possibly ranging from 1 to 100, and the number of glycerol groups possibly ranging from 2 to 30; or alternatively these compounds comprising at least one fatty chain comprising from 8 to 30 carbon atoms and especially from 16 to 30 carbon atoms.

Mention may also be made of alkoxylated alcohols, such as condensates of ethylene oxide and of propylene oxide with fatty alcohols; polyethoxylated fatty amides preferably having from 2 to 30 ethylene oxide units, polyglycerolated fatty amides comprising on average from 1 to 5, and in particular from 1.5 to 4, glycerol groups; ethoxylated fatty acid esters of sorbitan preferably containing from 2 to 40 ethylene oxide units, fatty acid esters of sucrose, polyoxyalkylenated and preferably polyoxyethylenated fatty acid esters containing from 2 to 150 mol of ethylene oxide, including oxyethylenated plant oils, N—(C6-24 alkyl)glucamine derivatives, amine oxides such as (C10-14 alkyl)amine oxides or N—(C10-14 acyl)aminopropylmorpholine oxides. Mention may also be made of PEG-40 hydrogenated castor oil.

Mention may also be made of nonionic surfactants of alkyl(poly)glycoside type, represented especially by the following general formula:

R₁O—(R₂O)ₜ-(G)ᵥ

wherein:
R₁ represents a linear or branched alkyl or alkenyl radical comprising 6 to 24 carbon atoms and especially 8 to 18 carbon atoms, or an alkylphenyl radical whose linear or branched alkyl radical comprises 6 to 24 carbon atoms and especially 8 to 18 carbon atoms;
R₂ represents an alkylene radical comprising 2 to 4 carbon atoms,
G represents a sugar unit comprising 5 to 6 carbon atoms,
t denotes a value ranging from 0 to 10 and preferably 0 to 4,
v denotes a value ranging from 1 to 15 and preferably 1 to 4.

Preferably, the alkylpolyglycoside surfactants are compounds of the formula described above in which:
R₁ denotes a linear or branched, saturated or unsaturated alkyl radical comprising from 8 to 18 carbon atoms,
R₂ represents an alkylene radical comprising 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C8/C16 alkyl(poly)glycosides 1,4, and especially decyl glucosides and caprylyl/capryl glucosides, are most particularly preferred.

Among the commercial products, mention may be made of the products sold by the company COGNIS under the names PLANTAREN® (600 CS/U, 1200 and 2000) or PLANTACARE® (818, 1200 and 2000); the products sold by the company SEPPIC under the names ORAMIX CG 110 and ORAMIX NS 10; the products sold by the company BASF under the name LUTENSOL GD 70, or else the products sold by the company CHEM Y under the name AG10 LK.

Preferably, use is made of C8/C16-alkyl(poly)glycosides 1,4, especially as an aqueous 53% solution, such as those sold by Cognis under the reference Plantacare® 818 UP.

Other examples of nonionic surfactants that may be used in the present disclosure comprise fatty acid alkanolamides (isopropanolamides of fatty acids) such as cocoamide MIPA, lauramide MIPA, Linoleamide MIPA, Myristamide MIPA, Oleamide MIPA, stearamide MIPA, isostearamide MIPA, and mixtures thereof.

Mention may also be made of nonionic surfactants chosen from PEG-40 hydrogented castor oil.

In certain embodiments. the nonionic surfactants of the present disclosure are chosen from (C6-24 alkyl)polyglycosides, and from fatty acid alkanolamides, and more particularly (C8-18 alkyl)(poly)glycosides, ethoxylated C8-C30 fatty acid esters of sorbitan, polyethoxylated C8-C30 fatty alcohols and polyoxyethylenated C8-C30 fatty acid esters, these compounds preferably containing from 2 to 150 mol of ethylene oxide, and mixtures thereof.

In preferred embodiments of the disclosure, the nonionic surfactants are chosen from coco-glucoside, cocamide MIPA, PEG-40 hydrogenated castor oil, and mixtures thereof.

In some embodiments, the nonionic surfactant in the composition of the present disclosure is coco-glucoside. In other embodiments, the nonionic surfactant comprises coco-glucoside and cocamide MIPA.

The total amount of nonionic surfactant present in the composition is from about 1% to 10% by weight, such as from about 1% to about 8% by weight, from about 2% to about 7% by weight, from about 2% to about 6% by weight, or from about 2% to about 5%, by weight of active material, based on the total weight of the composition, including all ranges and subranges therebetween.

In various embodiments, the total amount of nonionic surfactant is about 1%, 1.5%, 2%, 2.5%, 3%, 3.5%, 4%, 4.5%, 5%, 5.1%, 5.2%, 5.3%, 5.4%, 5.5%, 5.6%, 5.7%, 5.8%, 5.9%, 6%, 6.5%, 7%, 7.5%, 8%, 8.5%, 9%, 9.5%, and 10% by weight of active material, based on the total weight of the composition.

### Anionic Surfactant

The compositions of the present disclosure comprise at least one anionic surfactant.

The term “anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from —CO₂H, —CO₂⁻, —SO₃H, —SO₃⁻, —OSO₃H, —OSO₃⁻, —H₂PO₃, —HPO₃⁻, —PO₃²⁻, —H₂PO₂, ═HPO₂, —HPO₂⁻, ═PO₂⁻, ═POH, and ═PO⁻ groups.

The anionic surfactants may be sulfate, sulfonate and/or carboxylic (or carboxylate) surfactants, or mixtures thereof.

Sulfate anionic surfactants comprise at least one sulfate function but do not comprise any carboxylate or sulfonate functions. The sulfate anionic surfactants that may be used comprise at least one sulfate function (—OSO₃H or —OSO₃).

They may be chosen from the following compounds: alkyl sulfates, alkyl ether sulfates, alkylamido ether sulfates, alkylaryl polyether sulfates, monoglyceride sulfates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Preferentially, the sulfate anionic surfactants are chosen, alone or as a mixture, from:
alkyl sulfates, especially of C6-C24 or even C12-C20,
alkyl ether sulfates, especially of C6-C24 or even C12-C20, preferably comprising from 2 to 20 ethylene oxide units;

in particular in the form of alkali metal or alkaline-earth metal, ammonium or amino alcohol salts.

Sulfonate anionic surfactants comprise at least one sulfonate function (—SO₃H or —SO₃⁻) and may optionally also comprise one or more sulfate functions, but do not comprise any carboxylate functions. The sulfonate anionic surfactants that may be used comprise at least one sulfonate function (—SO₃H or —SO₃⁻).

They may be chosen from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Preferentially, the sulfonate anionic surfactants are chosen, alone or as a mixture, from:
C6-C24 and especially C12-C20 alkylsulfosuccinates, especially laurylsulfosuccinates;
C6-C24 and especially C12-C20 alkyl ether sulfosuccinates;
(C6-C24)acylisethionates and preferably (C12-C18)acylisethionates,

in particular in the form of alkali metal or alkaline-earth metal, ammonium or amino alcohol salts.

Carboxylate anionic surfactants comprise at least one carboxylic or carboxylate function (—COOH or —COO⁻) and may optionally also comprise one or more sulfate and/or sulfonate functions. The carboxylic anionic surfactants that may be used thus comprise at least one carboxylic or carboxylate function (—COOH or —COO⁻).

They may be chosen from the following compounds: acylglycinates, acyllactylates, acylsarcosinates, acylglutamates; alkyl-D-galactosideuronic acids, alkyl ether carboxylic acids, alkyl(C6-30 aryl) ether carboxylic acids, alkylamido ether carboxylic acids; and also the salts of these compounds; the alkyl and/or acyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Use may also be made of the C6-C24 alkyl monoesters of polyglycoside-polycarboxylic acids, such as C6-C24 alkyl polyglycoside-citrates, C6-C24 alkyl polyglycoside-tartrates and C6-C24 alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those comprising from 2 to 50 alkylene oxide and in particular ethylene oxide groups, such as the compounds sold by the company Kao under the name Akypo.

The polyoxyalkylenated alkyl(amido) ether carboxylic acids that may be used are preferably chosen from those of formula (1):

R₁—(OC₂H₄)ₙ—OCH₂COOA (1)

wherein:
R¹ represents a linear or branched C6-C24 alkyl or alkenyl radical, an alkyl(C8-C9)phenyl radical, a radical R2CONH—CH2-CH2— with R2 denoting a linear or branched C9-C21 alkyl or alkenyl radical, preferably, R1 is a C8-C20 and preferably C8-C18 alkyl radical, and aryl preferably denotes phenyl,
n is an integer or decimal number (average value) ranging from 2 to 24 and preferably from 2 to 10,
A denotes H, ammonium, Na, K, Li, Mg or a monoethanolamine or triethanolamine residue.

It is also possible to use mixtures of compounds of formula (1), in particular mixtures of compounds containing different groups R1.

The polyoxyalkylenated alkyl(amido) ether carboxylic acids that are particularly preferred are those of formula (1) in which:
R1 denotes a C12-C14 alkyl, cocoyl, oleyl, nonylphenyl or octylphenyl radical,
A denotes a hydrogen or sodium atom, and
n varies from 2 to 20 and preferably from 2 to 10.

Even more preferentially, use is made of compounds of formula (1) in which R denotes a C12 alkyl radical, A denotes a hydrogen or sodium atom and n ranges from 2 to 10.

Preferentially, the carboxylic anionic surfactants are chosen, alone or as a mixture, from:
acylglutamates, especially of C6-C24 or even C12-C20, such as stearoylglutamates, and in particular disodium stearoylglutamate;
acylsarcosinates, especially of C6-C24 or even C12-C20, such as palmitoylsarcosinates, and in particular sodium palmitoylsarcosinate;
acyllactylates, especially of C12-C28 or even C14-C24, such as behenoyllactylates, and in particular sodium behenoyllactylate;
C6-C24 and especially C12-C20 acylglycinates;
(C6-C24)alkyl ether carboxylates and especially (C12-C20)alkyl ether carboxylates;
polyoxyalkylenated (C₆-C₂₄)alkyl(amido) ether carboxylic acids,

in particular those comprising from 2 to 50 ethylene oxide groups; in particular in the form of alkali metal or alkaline-earth metal, ammonium or amino alcohol salts.

When the anionic surfactant is in salt form, the said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactants are chosen, alone or as a mixture, from:
C6-C24 and especially C12-C20 alkyl sulfates;
C6-C24 and especially C12-C20 alkyl ether sulfates; preferably comprising from 2 to 20 ethylene oxide units;
C6-C24 and especially C12-C20 alkylsulfosuccinates, especially laurylsulfosuccinates;
C6-C24 and especially C12-C20 alkyl ether sulfosuccinates;
(C6-C24)acylisethionates and preferably (C12-C18)acylisethionates;
C6-C24 and especially C12-C20 acylsarcosinates; especially palmitoylsarcosinates;
(C6-C24)alkyl ether carboxylates, preferably (C12-C20)alkyl ether carboxylates;
polyoxyalkylenated (C6-C24)alkyl(amido) ether carboxylic acids and salts thereof, in particular those comprising from 2 to 50 alkylene oxide and in particular ethylene oxide groups;
C6-C24 and especially C12-C20 acylglutamates;
C6-C24 and especially C12-C20 acylglycinates;

in particular in the form of alkali metal or alkaline-earth metal, ammonium or amino alcohol salts.

In particular, (C₁₂-C₂₀)alkyl sulfates, (C₁₂-C₂₀)alkyl ether sulfates comprising from 2 to 20 ethylene oxide units, especially in the form of alkali metal, ammonium, amino alcohol and alkaline-earth metal salts, or a mixture of these compounds, may be chosen. In at least one embodiment, sodium lauryl ether sulfate containing 2.2 mol of ethylene oxide is chosen.

The anionic surfactant is preferably present in the composition in an amount of from about 5% to 30% by weight, such as from about 5% to about 25% by weight, from about 5% to about 20% by weight, from about 8% to about 20% by weight, or from about 9% to about 20%, by weight of active material, based on the total weight of the composition, including all ranges and subranges therebetween.

In various embodiments, the total amount of anionic surfactant is about 5%, 6%, 7%, 8%, 9%, 9.8%, 10%, 10.1, 10.5%, 11%, 11.5%, 12%, 12.5%, 13%, 13.5%, 13.7%, 14%, 14.5%, 15%, 16%, 17%, 17.5%, 18%, 20%, 22%, 24%, 25%, 26%, 27%, and 30% by weight of active material, based on the total weight of the composition.

In some embodiments of the present disclosure, the total amount of amphoteric surfactants is about 20% or more by weight, such as about 21%, or about 22%, or about 23%, or about 24%, or about 25%, or about 26%, or about 27%, or about 28%, or about 29% or about 30% by weight of active material, based on the total weight of the surfactant mixture of the composition of the disclosure. Without wishing to be bound to any one theory, it is believed the amount of the amphoteric surfactants as described contributes to the clarity of the compositions of the disclosure in the presence of the ampholytic polymer (a) and the copolymer (b) of the disclosure.

In other embodiments of the present disclosure, the total amount of nonionic surfactants is about 8% or more by weight, such as about 9%, or about 9.9%, or about 10%, or about 11%, or about 12%, or about 13%, or about 13.6%, or about 14%, or about 15% or about 16%, or about 16.7%, or about 17%, or about 18%, or about 19% or about 20% by weight of active material, based on the total weight of the surfactant mixture of the composition of the disclosure. Without wishing to be bound to any one theory, it is believed the amount of the nonionic surfactants as described contributes to the clarity of the compositions of the disclosure in the presence of the silane of the disclosure.

### Water

The compositions according to various embodiments of the disclosure may be aqueous. Water can be present in amounts of about 95% or less, such as from about 95% to about 5% by weight, or about 90%, 80%, 70%, 60%, 50%, 40%, 30%, 20%, 10%, or 5% or less, by weight, based on the total weight of the composition. In further embodiments, water can be present in an amount of about 95%, such as about 90%, 80%, 70%, 60%, 50%, 40%, 30%, 20%, 10%, or 5%, by weight, based on the total weight of the composition, including all ranges and subranges therebetween. Additionally, water can be present in the compositions of the present disclosure in the amount of from about 20% to about 95% by weight, from about 40% to about 90% by weight, or from about 50% to about 80% by weight, based on the total weight of the compositions.

In other embodiments, water can be present in the compositions of the present disclosure in the amount of at least about 95%, 90%, 80%, 70%, 60%, 50%, 45%, 40%, 30%, 20%, 10%, 5% by weight, based on the total weight of the compositions.

pH

The pH of the compositions according to the disclosure generally ranges from about 4 to about 7, for example from about 5 to about 6.5, or from about 5 to about 6.0, or from about 5 to about 5.5, including ranges and subranges therebetween. In certain embodiments, the pH of the compositions according to the disclosure is at about 5, 5.1, 5.2, 5.3, 5.4, 5.5, 5.6, 5.7, 5.8, 5.9, and 6.

### Additional Components

The composition according to the disclosure may also comprise additives chosen from anionic polymers, nonionic polymers, rheology modifiers, thickening and/or viscosity modifying agents, associative or non-associative polymeric thickeners, non-polymeric thickeners, non-polymeric cationic surfactants, nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, pH stabilizers and solvents, and mixtures thereof. A person skilled in the art will take care to select the optional additives and the amount thereof such that they do not harm the properties of the compositions of the present disclosure.

If present in the composition, these additives are generally present in an amount ranging up to about 40% by weight of active material relative to the total weight of the composition, such as up to about 30%, up to about 20%, up to about 15%, up to about 10%, up to about 5%, such as from 0% to 20%.

The compositions of certain embodiments may comprise stabilizers, for example sodium chloride, magnesium dichloride or magnesium sulfate.

The rheology modifiers and thickening/viscosity-modifying agents that may be employed in compositions of the present disclosure may include any water-soluble or water-dispersible compound that is compatible with the compositions of the disclosure, such as acrylic polymers, non-acrylic polymers, starch, cellulose-based polymers, non-polymeric and polymeric gelling agents, and mixtures thereof.

The compositions may be packaged in various forms, especially in bottles, in pump bottles or in aerosol containers so as to apply the composition in vaporized form or in the form of a mousse. The compositions may also impregnate applicators, especially gloves or wipes.

The composition may be applied by hand, with an applicator nozzle, with a container equipped with a pump and a dispensing comb, or with an insoluble substrate impregnated with the composition.

### Processes/Methods

The compositions according to the disclosure may be prepared according to techniques that are well known to those skilled in the art.

Embodiments of the disclosure also relate to a process for washing keratinous materials, such as hair, which consists in applying an effective amount of a composition as defined above to the said keratinous materials, and in rinsing, for example with water, after an optional leave-on time.

Certain embodiments also relate to a process for cleansing keratinous materials, which consists in applying an effective amount of a composition as defined above to the said keratinous materials, and in optionally rinsing, for example with water, after an optional leave-on time.

In some embodiments, keratinous materials, such as hair, may be washed or cleansed by a first step of applying the composition of the disclosure onto hair, with an optional leave-on time, followed by a second step of applying a conditioning composition, with an optional step of rinsing the hair with water between the two first and second steps, and optionally rinsing the conditioning composition, for example with water, after an optional leave-on time.

The compositions may be applied to keratinous substrates, such as the hair, and subsequently rinsed off. In various embodiments, the compositions comprise shampoo compositions for shampooing and/or conditioning the hair, and in various embodiments the shampoo composition will traditionally be rinsed off the hair within a short period of time after application to the hair, such as a period of time up to about 10 minutes, up to about 5 minutes, or up to about 2 minutes after application to the hair.

In various embodiments, processes according to the disclosure comprise applying the compositions described onto keratinous substrates, such as the hair, and subsequently rinsing the compositions off. The processes may, in various embodiments, impart volume to the keratinous substrate to which the composition is applied, even after the composition is rinsed off. The processes may additionally impart long lasting volume as well as conditioning to the keratinous substrates.

As used herein, the method/process and composition disclosed herein may be used on the hair that has not been artificially dyed, pigmented or permed.

As used herein, the method/process and composition disclosed herein may be also used on the hair that has been artificially dyed, pigmented or permed.

### Examples

The following Examples are intended to be non-restrictive and explanatory only, with the scope of the disclosure being defined by the claims.

The ingredient amounts in the compositions/formulas described below are expressed in % by weight, based on the total weight of the composition/formula.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements. The following examples are intended to illustrate the disclosure without limiting the scope as a result.

### Example 1: Compositions

TABLE 1
Shampoo Compositions (Inventive compositions)

| | % by Weight | | | | |
|---|---|---|---|---|---|
| | (Active Matter) | | | | |
| | FORMULA | | | | |
| INCI US NAME | A | B | C | D | E |
| POLYQUATERNIUM- | 0.103 | 0.103 | 0.103 | 0.103 | 0.103 |
| 53^{a} | | | | | |
| POLYQUATERNIUM- | 0.05 | 0.05 | 0.05 | 0.05 | 0.05 |
| 68^{b} | | | | | |
| AMINOPROPYL | 0.75 | 0.75 | 0.75 | 0.75 | 0.75 |
| TRIETHOXYSILANE^{c} | | | | | |
| COCO-BETAIN^{d} | 5.61 | 5.61 | 5.61 | 5.61 | 5.61 |
| PEG-40 | — | | — | 0.5 | 0.7 |
| HYDROGENATED | | | | | |
| CASTOR OIL | | | | | |
| (100% by weight | | | | | |
| active) | | | | | |
| COCAMIDE | — | — | 1 | 2 | 2 |
| MIPA^{e} | | | | | |
| COCO- | 2.08 | 2.08 | 2.08 | 2.08 | 2.08 |
| GLUCOSIDE^{f} | | | | | |
| SODIUM | 9.8 | 9.8 | 14 | 14 | 14 |
| LAURETH | | | | | |
| SULFATE^{g} | | | | | |
| SODIUM | 3.35 | 3.35 | — | — | — |
| LAUROYL | | | | | |
| METHYL | | | | | |
| ISETHIONATE^{h} | | | | | |
| WATER, AND AS | QS | QS | QS | QS | QS |
| DESIRED OR | | | | | |
| NEEDED - | | | | | |
| ORGANIC | | | | | |
| SOLVENTS AND | | | | | |
| ADDITIVES | | | | | |
| SUCH AS | | | | | |
| PRESERVATIVES, | | | | | |
| pH | | | | | |
| ADJUSTERS, | | | | | |
| FRAGRANCE | | | | | |

^{a}commercially available under the tradename MERQUAT 2003PR POLYMER from Nalco-Lubrizol, 20% by weight of active matter
^{b}commercially available under the tradename LUVIQUAT SUPREME AT1 from BASF, 20% by weight of active matter
^{c}commercially available under the tradename SILSOFT A-1100 from Momentive Performance Materials or under the tradename KBE-903, from Shinetsu
^{d}commercially available as DEHYTON AB 30 from BASF or GENAGEN KB, commercially available from Clariant, 30% by weight of active matter
^{e}commercially available underthe tradename EMPILAN CIS xxx fromHuntsmanxxx
^{f}commercially available under the tradename PLANTACARE 818 UP from BASF, 52% by weight active matter
^{g}commercially available under the tradename TEXAPON AOS 225 UP from BASF, 70% by weight active matter
^{h}commercially available under the tradename ISELUX LQ-CLR-SB from Innospec Active Chemicals, 33.5% by weight active matter

The Shampoo Formulas in Table 1 above were prepared according to the following protocol

1) Water was first added to the beaker.
2) Coco-glucoside was added to the beaker and mixed until uniform.
3) Aminopropyl Triethoxysilane was added to the mixture. The pH was then adjusted to 5.4 with Citric Acid.
4) Polyquaternium-53 and 50% of total Coco-Betaine were added to the mixture in small amounts. The solution was stirred in between additions.
5) Polyquaternium-68 and the rest of total Coco-Betaine were added to the mixture in small amounts. The solution was stirred in between additions.
6) If there is cocamide MIPA in the formula, then it is added in a separate beaker and diluted by 15% of total water and heated to 65° C. The solution was added and stirred into the mixture in step 5.
7) If sodium lauroyl methyl isethionate is present in the formula, then it was added at this stage to the mixture in step 5.
8) Sodium Laureth Sulfate was added to the mixture and stirred until uniform.
9) Additives such as preservatives and pH adjusters were added to the mixture in small amounts. The solution was stirred in between additions.
10) Fragrance, if desired, was added and stirred.

The inventive shampoos prepared according to the above protocol were clear or transparent, The word “clear” or “transparent” as used herein means that the light can pass through the inventive composition such that objects behind it are distinctly visible to the naked eye

TABLE 2
Conditioner composition (rinse-out), Formula F, for treating hair after
shampooing hair with the inventive composition

| | FORMULA F |
|---|---|
| | % by Weight |
| INCI US NAME | (Active Matter) |
| CETEARYL ALCOHOL^{a} | 2 |
| AMINOPROPYL TRIETHOXYSILANE^{b} | 0.5 |
| POLYQUATERNIUM-11^{c} | 0.05 |
| HYDROXYETHYLCELLULOSE^{d} | 0.3 |
| POLYQUATERNIUM-37 (and) MINERAL OIL (and) | 1.1 |
| PPG-1 TRIDECETH-6^{e} | |
| PEG-150/STEARYL ALCOHOL/SMDI COPOLYMER^{f} | 0.52 |
| WATER, AND AS DESIRED OR NEEDED - | QS |
| ORGANIC SOLVENTS AND ADDITIVES SUCH AS | |
| PRESERVATIVES, pH ADJUSTERS, FRAGRANCE | |

^{a}commercially available under the tradename LANETTE O OR from BASF
^{b}commercially available under the tradename SILSOFT A-1100 from Momentive Performance Materials or under the tradename KBE-903, from Shinetsu
^{c}commercially available under the tradename GAFQUAT 755 from ISP (ASHLAND), 20% by weight of active matter
^{d}commercially available as NATROSOL 250 HHR PC or NATROSOL 250 HHR CS from Ashland
^{e}commercially available under the tradename SALCARE SC 95 from BASF, 50% by wt polyquaternium-37, 35% by wt mineral oil, and 6% by wt PPG-1 trideceth-6 active matter
^{f}commercially available under the tradename ACULYN 46N POLYMER from Rohm & Haas (Dow Chemical), 13% by weight active matter

TABLE 3
Comparative Commercial Shampoo Formula G
(advertised for its volumizing properties)
INCI US

WATER
SODIUM LAURYL SULFATE
SODIUM LAURETH SULFATE
COCAMIDOPROPYL BETAINE
SODIUM CITRATE
SODIUM XYLENESULFONATE
FRAGRANCE
SODIUM CHLORIDE
CITRIC ACID
SODIUM BENZOATE
HYDROXYPROPYL METHYLCELLULOSE
TETRASODIUM EDTA
TRISODIUM ETHYLENEDIAMINE
DISUCCINATE
PANTHENOL
PANTHENYL ETHYL ETHER
METHYLCHLOROISOTHIAZOLINONE
METHYLISOTHIAZOLINONE

TABLE 4
Commercial conditioner composition (rinse-out), Formula H, for
treating hair after shampooing hair with the commercial
comparative composition (advertised for its volumizing properties)
INCI US

WATER
CETYL ALCOHOL (C16)
STEARAMIDOPROPYL DIMETHYLAMINE
STEARYL ALCOHOL (C18)
QUATERNIUM 18
FRAGRANCE
BIS AMINOPROPYL DIMETHICONE
BENZYL ALCOHOL
CETARYL ALCOHOL
HYDROXYPROPYL GUAR
GLYCERYL STEARATE
CITRIC ACID
POLYSORBATE 60
EDTA
PANTHENOL
PANTHENYL ETHYL ETHER
OLEYL ALCHOL
TRISODIUM ETHYLENEDIAMINE
DISUCCINATE
METHYLCHLOROISOTHIAZOLINONE
METHYLISOTHIAZOLINONE

### Example 2: Shampoo Tests

The inventive shampoo Formula C and comparative commercial shampoo Formula G were tested on the hair of the heads of 12 human panelists in a salon. Panelists consisted of women with very fine to fine hair diameter, lightly sensitized, and medium to long hair length. Both formulas were applied and evaluated on the same person, with the inventive shampoo formula applied to half of the head, and the comparative commercial shampoo formula applied to the other half of the head for each human panelist. It was found that the inventive shampoo formula performed statistically significantly better than the comparative commercial shampoo formula with respect to the following attributes: volume, mass, body, discipline, shape, smooth feel, ease of combing, with a creamy compact foam. Another inventive shampoo formula, Formula A was compared to comparative commercial shampoo formula G; It was found that the inventive shampoo formula showed an improvement in attributes such as volume, ease of dry combing, suppleness, bounce, model preference, ease of passing fingers in wet state with creamy and compact foam characteristics against the comparative commercial shampoo formula.

### Example 3: Shampoo/Conditioner Tests

According to one embodiment of the disclosure, inventive shampoo Formula B, was compared to comparative commercial shampoo Formula G using a shampoo-conditioner regimen (“bundle” system) wherein inventive shampoo Formula B and conditioner Formula F (“inventive bundle”), and comparative commercial shampoo Formula G and conditioner Formula H (“comparative bundle”) were tested on the hair of the heads of 12 human panelists in a salon in a half head study.

The inventive bundle performed statistically significantly better than the comparative bundle in key performance attributes such as mass, body, shape, smooth feel and ease of combing.

### Example 4 APTES Deposition

Long term APTES deposition was studied by Plasma Optical Emission Spectrometer; the deposition amount of elemental silicon was measured as an indication of APTES deposition.

Protocol: medium bleached hair swatches were washed with a conventional shampoo and then rinsed for 30 seconds. Next 0.4 g of each test formulation per gram of hair was applied to a hair swatch, lathered for 30 seconds, and allowed to rest for one minute. The swatches were then rinsed with water for 30 seconds. As required for successive washings, the test formulations and rinses were repeated until 5 or 10 applications were reached. Samples were allowed to air dry after all required applications were complete. It is important to note that the APTES molecule is the only known contributor of silicon in all of the formulations.

TABLE 5

| | Number of | |
|---|---|---|
| Same Day | Wash | Silicon Deposition |
| measurement | Cycles | ppm |
| | 0 | 204 |
| | 1 | 238 |
| | 5 | 355 |
| | 10 | 363 |

TABLE 6
Formula containing 0.75% by weight of APTES (3-AMINOPROPYL
TRIETHOXYSILANE) tested for deposition studies

| | Formula I |
|---|---|
| | % by Weight |
| INCI US | (Active Matter)* |
| POLYQUATERNIUM-53 | 0.1 |
| AMINOPROPYL TRIETHOXYSILANE | 0.75 |
| COCO-BETAINE | 5.61 |
| SODIUM LAURETH SULFATE | 9.8 |
| SODIUM LAUROYL METHYL ISETHIONATE | 3.35 |
| WATER, AND AS DESIRED OR NEEDED - | QS |
| ORGANIC SOLVENTS AND ADDITIVES SUCH | |
| AS PRESERVATIVES, pH ADJUSTERS, | |
| FRAGRANCE | |

*Activities of ingredients based on information in Table 1

The results in Table 5 show that significant levels of silicon deposition are reached by the 5th wash cycle with no significant build up upon further use. This indicates that APTES deposition is a gradual process upon repeated application of the product and hence creates full impact after several wash cycles and at the same time, does not wash off immediately and has a long lasting effect.

It is to be understood that the foregoing describes preferred embodiments of the disclosure and that modifications may be made therein without departing from the spirit or scope of the disclosure as set forth in the claims.

What is claimed is:

1. A rinse-off composition for cleansing keratin substrates, the composition comprising:
(a) at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
(b) at least one copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole;
(C) at least one silane corresponding to formula (I):
R₁Si(OR₂)_{z}(R₃)ₓ(OH)_{y} (I)
in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3; and
(d) a surfactant mixture comprising:
i. at least one amphoteric surfactant;
ii. at least one nonionic surfactant; and
iii. at least one anionic surfactant;
wherein the total amount of amphoteric surfactants is about 20% or more by weight, based on the total weight of the surfactant mixture; and
(e) water.

2. The composition according to claim 1, wherein the cationic charge density of the ampholytic polymer ranges from greater than 1 meq/g to about 3.5 meq/g.

3. The composition according to claim 2, characterized in that the ampholytic polymer comprises from 30 mol percent to 70 mol percent of units derived from a monomer of (meth)acrylamide type, from 10 mol percent to 60 mol percent of units derived from a monomer of (meth)acrylamidoalkyltrialkylammonium type and from 1 mol percent to 20 mol percent of units derived from an acidic monomer of (meth)acrylic acid type.

4. The composition according to claim 3, characterized in that the ampholytic polymer comprises units derived from the following monomers: (i) acrylamide, (ii) methacrylamidopropyltrimethylammonium chloride, and (iii) acrylic acid.

5. The composition according to claim 4, wherein the at least one ampholytic polymer is an ampholytic terpolymer of methacrylamidopropyltrimethylammonium chloride (MAPTAC), acrylamide, and acrylic acid.

6. The composition according to claim 5, wherein the at least one ampholytic polymer is Polyquaternium-53.

7. The composition according to claim 4, wherein the amount of the ampholytic polymer ranges from about 0.05% to about 0.4% by weight, based on the total weight of the composition.

8. The composition according to claim 1, wherein the monomer (i) of the at least one copolymer (b) is selected from methacrylic acid amide, monomer (ii) of the at least one copolymer (b) is selected from N-vinylpyrrolidone and monomer (iii) of the at least one copolymer (b) is selected from 3-alkyl-1-vinylimidazolium salts with physiologically acceptable anions.

9. The composition according to claim 8, wherein monomer (iii) is 3-methyl-1-vinylimidazolium methyl sulfate.

10. The composition according to claim 9, wherein the at least one copolymer (b) is Polyquaternium-68.

11. The composition according to claim 8, wherein the amount of the copolymer (b) ranges from about 0.005% to about 0.2% by weight, based on the total weight of the composition.

12. The composition according to claim 1, wherein the silane of formula (I) is selected from 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy)propyltrimethoxysilane, p-aminophenyltrimethoxysilane and N-(2-aminoethylaminomethyl)phenethyltrimethoxysilane, their oligomers, and mixtures thereof.

13. The composition according to claim 12, wherein the silane of formula (I) is selected from 3-aminopropyltriethoxysilane, oligomers thereof, and mixtures thereof.

14. The composition according to claim 12, wherein the amount of the silane of formula (I) ranges from about 0.05% to less than 1% by weight, based on the total weight of the composition.

15. The composition according to claim 1, wherein the at least one amphoteric surfactant is selected from (C8-C20)alkylbetaines, sulfobetaines, (C8-C20)alkylsulfobetaines, (C8-C20)alkylamido(C1-C6)alkylbetaines, such as cocamidopropylbetaine, and (C8-C20)alkylamido(C1-C6)alkylsulfobetaines, and mixtures thereof.

16. The composition according to claim 1, wherein the at least one nonionic surfactant is selected from alkyl(poly)glycosides, fatty acid alkanolamides, alkoxylated alcohols, and mixtures thereof.

17. The composition according to claim 16, wherein the at least one nonionic surfactant is selected from decyl glucoside, cocamide MIPA, PEG-40 hydrogenated castor oil, and mixtures thereof.

18. The composition according to claim 1, wherein the at least one anionic surfactant is selected from sodium laureth sulfate, sodium lauryl sulfate, sodium lauroyl methyl isethionate, and mixtures thereof.

19. The composition according to claim 1, wherein the total amount of nonionic surfactants is about 8% or more by weight, based on the total weight of the surfactant mixture in the composition.

20. The composition according to claim 1, wherein the composition is clear.

21. The composition according to claim 1, wherein the keratin substrates include hair.

22. A rinse-off composition for cleansing keratin substrates, the composition comprising:
(a) from about 0.05% to about 0.4% by weight, based on the total weight of the composition, of at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
(b) from about 0.005% to about 0.2% by weight, based on the total weight of the composition, of at least one copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole;
(C) from about 0.05% to less than 1% by weight, based on the total weight of the composition, of at least one silane corresponding to formula (I):
R₁Si(OR₂)_{z}(R₃)ₓ(OH)_{y} (I)
in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3; and
(d) a surfactant mixture comprising:
i. at least one amphoteric surfactant;
ii. at least one nonionic surfactant; and
iii. at least one anionic surfactant;
wherein the total amount of amphoteric surfactants in the surfactant mixture is about 20% or more by weight, based on the total weight of the composition; and
(e) water.

23. A method for washing, cleansing, and/or rinsing a keratin substrate, the method comprising:
contacting the keratin substrate with a composition comprising:
(a) at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
(b) at least one copolymer comprising a monomer (i) selected from acrylic acid amide, methacrylic acid amide, N-alkylacrylic acid amide and N-alkyl-methacrylic acid amide; a monomer (ii) selected from N-vinyllactams and N-vinylpyrrolidone; a monomer (iii) selected from quaternized N-vinylimidazoles; and the monomer N-vinylimidazole;
(C) at least one silane corresponding to formula (I):
R₁Si(OR₂)_{z}(R₃)ₓ(OH)_{y} (I)
in which:
R₁ is a cyclic or acyclic, linear or branched, saturated or unsaturated C₁-C₂₂, in particular C₂-C₂₀, hydrocarbon-based chain, which may be substituted with a group chosen from amine groups NH₂ or NHR (R being a linear or branched C₁-C₂₀, in particular C₁-C₆, alkyl, a C₃-C₄₀ cycloalkyl or a C₆-C₃₀ aromatic radical); the hydroxyl group (OH), a thiol group, an aryl group (more particularly benzyl), which is possibly substituted with an NH₂ or NHR group; it being possible for R1 to be interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2, with z+x+y=3; and
(d) a surfactant mixture comprising:
i. at least one amphoteric surfactant;
ii. at least one nonionic surfactant; and
iii. at least one anionic surfactant;
wherein the total amount of amphoteric surfactants in the surfactant mixture is about 20% or more by weight, based on the total weight of the composition; and
(e) water.

24. The method according to claim 23, wherein the keratin substrate is hair.

25. The method according to claim 24, further comprising leaving the composition on the keratin substrate for a leave-on time and then rinsing the keratin substrate with water.

26. The method according to claim 25, further comprising applying a conditioning composition onto the keratin substrate.

27. The method according to claim 26, further comprising leaving the conditioning composition on the keratin substrate for a leave-on time and then rinsing the keratin substrate with water.
